(19) United States
(12) Patent Application Publication
(10) Pub. No.: US 2017/0181649 A1
(43) Pub. Date: Jun. 29, 2017

(54) Systems and Methods for Determining Blood Pressure

(71) Applicant: Amiigo, Inc., Palo Alto, CA (US)

(72) Inventors: Abraham Carter, Palo Alto, CA (US); Sameer Sontakey, Palo Alto, CA (US); Muneem Shahriar, Palo Alto, CA (US)

(21) Appl. No.: 15/388,823

(22) Filed: Dec. 22, 2016

Related U.S. Application Data
(60) Provisional application No. 62/271,622, filed on Dec. 28, 2015.

(51) Int. Cl. A61B 5/024; A61B 5/00

(52) U.S. Cl. CPC A61B 5/02416; A61B 5/0002; A61B 5/0059; A61B 5/6824

(57) ABSTRACT

Systems and methods for determining a blood pressure of a subject from a photoplethysmogram are disclosed. The systems and methods include acquiring raw photoplethysmogram (PPG) data from a subject by measuring light reflected from or transmitted through a portion of the subject's body, determining a mean beat shape from the raw PPG data, and analyzing the mean beat shape to determine a blood pressure of the subject. The method can include analyzing a distinct shape of the mean beat shape to determine the blood pressure. The method can include identifying individual beats within the raw PPG data, collecting motion data and filtering the individual beats against the motion data, measuring biometric features of filtered beats, scaling individual beats in time and amplitude, and measuring additional shape features of scaled beats.

### CROSS-REFERENCE TO RELATED APPLICATIONS

This application claims priority to U.S. Provisional Patent Application No. 62/271,622 which has a filing date of Dec. 28, 2015 and is titled “Systems and Methods for Determining Blood Pressure”.

### BACKGROUND

Blood pressure is an important indicator of an individual's cardiac health and can reveal potential and/or ongoing health problems. Medical professionals recommend regular and accurate blood pressure screenings to help prevent, diagnose, and monitor certain types of health problems related to blood pressure. For example, high blood pressure (hypertension) can greatly increase an individual's risk of heart disease and stroke. Likewise, low blood pressure (hypotension) can also have underlying causes such as serious heart, endocrine, or neurological disorders. Additionally, certain medications can cause abnormal blood pressures that can lead to deleterious health problems. Therefore, it is important to determine and/or monitor blood pressure in the individual.

Conventional methods for determining blood pressure include the use of a device such as a sphygmomanometer to measure the blood pressure of the individual. In general, the sphygmomanometer comprises an inflatable cuff and a manometer. The inflatable cuff is fitted around an arm of the individual and inflated with pressure to slowly collapse an artery. The pressure is slowly released until blood just begins to flow through the artery. The pressure is measured at this point with the manometer and recorded as the systolic blood pressure (usually in mm of Hg). The pressure is then slowly released again until blood begins to flow freely through the artery. The pressure at the point when the blood begins to flow freely is measured with the manometer and is recorded as the diastolic blood pressure (usually in mm of Hg). In some cases, blood pressure can be determined in this manner by a medical practitioner manually inflating the cuff and manually listening to the flow of the blood. In other cases, automatic devices can be used to determine blood pressure either by the method indicated above or by automatically inflating the cuff with pressure and releasing the pressure to measure blood pressure by evaluating the oscillations of the arteries.

Although conventional methods for determining blood pressure provide a variety of benefits, these methods are not without limitations. Some examples of these shortcomings include the need for an inflatable cuff that must be inflated and deflated to determine the blood pressure. Also, the inflatable cuff must be placed on an arm or similar extremity to carry out the measurement. Moreover, the inflatable cuff can be difficult to place and/or can be uncomfortable for the wearer. Additionally, the inflatable cuff can be bulky and can be difficult and/or uncomfortable to wear while sleeping and/or performing other activities such as walking or exercising. Another limitation is that these conventional techniques do not allow for continuous measurement of blood pressure because the arm or extremity must be allowed to rest between measurements (often up to fifteen minutes between measurements). Yet another limitation is that the process of determining blood pressure using conventional methods can create apprehension in some individuals. This apprehension can cause an increase in blood pressure and can lead to a measurement that is above the individual's normal blood pressure.

Thus, while a variety of conventional methods to determine blood pressure currently exist, challenges still exist, include the limitations listed above. Accordingly, it would be an improvement in the art to augment or even replace current techniques with other systems and methods.

### BRIEF SUMMARY

Described herein are some embodiments of systems and methods for determining a blood pressure of a subject from a photoplethysmogram. In some embodiments, the methods include acquiring raw photoplethysmogram (PPG) data from a subject by measuring light reflected from or transmitted through a portion of the subject's body, determining a mean beat shape from the raw PPG data, and analyzing the mean beat shape to determine a blood pressure of the subject. The methods can also include acquiring raw PPG data from a subject by acquiring individual pulse wave forms on a millisecond scale. The methods can also include analyzing a distinct shape of the mean beat shape to determine the blood pressure. The methods can also include identifying individual beats within the raw PPG data, collecting motion data and filtering the individual beats against the motion data, measuring biometric features of filtered beats, scaling individual beats in time and amplitude, and measuring additional shape features of scaled beats.

In some embodiments a method for beat shape analysis comprises determining a mean beat shape from raw photoplethysmogram (PPG) data, analyzing one or more of a biometric feature and an additional shape feature corresponding to the mean beat shape, and determining a blood pressure based at least in part on the analysis of the mean beat shape. The methods can also include acquiring raw photoplethysmogram (PPG) data from a subject by measuring light reflected from or transmitted through a portion of the subject's body, identifying individual beats within the raw PPG data by identifying peaks in the raw PPG data, identifying valleys in the raw PPG data, using the valleys to generate a base of the raw PPG data, and subtracting the base from the raw PPG data, filtering of the individual beats, scaling the individual beats in time and amplitude, analyzing the mean beat shape by correlating biometric features corresponding to a systolic portion of the mean beat shape, analyzing the mean beat shape by correlating biometric features corresponding to a diastolic portion of the mean beat shape, and/or classifying the determined blood pressure as one of hypotensive blood pressure, normotensive blood pressure, or hypertensive blood pressure.

In some embodiments, systems for determining blood pressure comprise a wearable device configured to acquire raw photoplethysmogram (PPG) data from a subject by measuring light reflected from or transmitted through a portion of the subject's body, a biometric engine configured to generate scaled beats and biometric features from the raw PPG data, and a blood pressure engine configured to analyze scaled beats, biometric features, and additional beat shape features to determine the subject's blood pressure. In some instances, the system is configured to determine the subject's blood pressure within an accuracy of 10 mm of Hg. In other instances, the system is configured to continuously monitor the subject's blood pressure in real-time. In yet other instances, the system is configured to continuously monitor increases and decreases in the subject's blood pressure.

### BRIEF DESCRIPTION OF THE DRAWINGS

In order to describe the manner in which the above-recited and other advantages and features of the disclosure can be obtained, a more particular description of the disclosure briefly described above will be rendered by reference to specific embodiments thereof which are illustrated in the appended drawings. Understanding that these drawings depict only typical embodiments of the disclosure and are not therefore to be considered to be limiting of its scope, the disclosure will be described and explained with additional specificity and detail through the use of the accompanying drawings in which:

FIG. 1 illustrates an example diagram of a system configured to determine blood pressure;

FIG. 2 illustrates example of a method for beat shape analysis to determine blood pressure;

FIG. 3 illustrates a wearable device that can be configured to implement embodiments of the disclosed systems and methods;

FIG. 4 illustrates an example circuit diagram for implementing embodiments of the disclosed methods and systems;

FIG. 5A illustrates an example of a PPG that can be generated by the light sensor of the circuit depicted in FIG. 2;

FIG. 5B illustrates an example of a hat that can be calculated for the PPG of FIG. 5A;

FIG. 5C illustrates an example of a base that can be calculated for the PPG of FIG. 5A;

FIG. 5D illustrates an example of beat data that is generated by subtracting the base from the PPG;

FIG. 5E illustrates an example of a smoothed and detrended beat within the beat data of FIG. 5D;

FIG. 6 illustrates some embodiments of a first beat mode;

FIG. 7 illustrates some embodiments of a second beat mode;

FIG. 8 illustrates some embodiments of a third beat mode;

FIG. 9 illustrates some embodiments of a fourth beat mode;

FIG. 10 illustrates a linear relationship modeled between an area_sum additional feature and a known systolic blood pressure;

FIG. 11 illustrates a linear relationship modeled between an amp_sum additional feature and a known systolic blood pressure;

FIG. 12 illustrates a linear relationship modeled between a deg.rsu biometric feature and a known systolic blood pressure;

FIG. 13 illustrates a linear relationship modeled between a pp.dvol biometric feature and a known systolic blood pressure;

FIG. 14 illustrates a linear relationship modeled between an i.sru biometric feature and a known systolic blood pressure;

FIG. 15 illustrates a linear relationship modeled between a pp.sp/dp biometric feature and a known diastolic blood pressure;

FIG. 16 illustrates a linear relationship modeled between an area_sum additional feature and a known diastolic blood pressure;

FIG. 17 illustrates a linear relationship modeled between an amp_10 additional feature and a known diastolic blood pressure;

FIG. 18 illustrates a linear relationship modeled between an amp_sum additional feature and a known diastolic blood pressure;

FIG. 19 illustrates a linear relationship modeled between an amp_dif additional feature and a known diastolic blood pressure;

FIG. 20 illustrates a linear relationship modeled between deg.sru biometric feature and a known mean arterial pressure (MAP) blood pressure;

FIG. 21 illustrates a linear relationship modeled between an area_sum additional feature and a known MAP blood pressure;

FIG. 22 illustrates a linear relationship modeled between a pp.vol biometric feature and a known MAP blood pressure;

FIG. 23 illustrates a linear relationship modeled between an amp.slope.srd biometric feature and a known MAP blood pressure; and

FIG. 24 illustrates a linear relationship modeled between a pp.sp/dp biometric feature and a known MAP blood pressure.

### DETAILED DESCRIPTION

The present disclosure relates to blood pressure. More particularly, some embodiments of the present disclosure relate to methods and systems for determining blood pressure with beat shape analysis. While the methods and systems can comprise any suitable component or step, in some cases, they can include collecting raw photoplethysmogram (PPG) and accelerometer data, processing the raw data with a biometric engine configured to perform signal processing to generate scaled beats and biometric features, processing the scaled beats and biometric features with a blood pressure engine configure to perform beat shape analysis, measure additional shape features, and model with machine learning to determine blood pressure.

In some embodiments, the methods and systems include providing a wearable device, collecting raw data, identifying individual beats, filtering individual beats, measuring biometric features, scaling filtered beats, analyzing beat shape, measuring additional shape features, modeling with machine learning to determine blood pressure, and correlating blood pressure with hypotension, hypertension, and/or normotension. In other embodiments, the methods and systems comprise determining and representing an individual's blood pressure in a medically accepted standard such as in millimeters of mercury (mm of Hg).

In the disclosure and in the claims, the term blood pressure (and variations thereof) may be used to refer to a pressure exerted by circulating blood upon walls of blood vessels. Blood pressure can refer to arterial pressure in systemic circulation. Blood pressure can also refer to systolic (or maximum) pressure over diastolic (or minimum) pressure. In some cases, blood pressure can also refer to mean arterial pressure (MAP) which is the average over a cardiac cycle and can be determined by cardiac output (CO), systemic vascular resistance (SVR), and central venous pressure (CVP). MAP can also be estimated from the systolic and diastolic pressures. In some instances, blood pressure can refer to pulse pressure which is the difference between the systolic and diastolic pressures.

In the disclosure and in the claims, low blood pressure can be referred to as hypotensive blood pressure or hypotension. In some cases, hypotension can refer to a disease state that causes or is caused by low blood pressure. Likewise, high blood pressure can be referred to as hypertensive blood pressure or hypotension. In some instances, hypertension can refer to a disease state that causes or is caused by high blood pressure. Normal blood pressure can be referred to as normotensive pressure or normotension. In some embodiments, normotensive pressure varies with age of the subject or individual. For example, infants from 1 to 12 months of age can have a normotensive blood pressure of 75-100 mm of Hg systolic and 50-70 mm of Hg diastolic, children from 1 to 5 years of age can have a normotensive blood pressure of 80-110 mm of Hg systolic and 50-80 mm of Hg diastolic, children from 6 to 12 years of age can have a normotensive blood pressure of 85-120 mm of Hg systolic and 50-80 mm of Hg diastolic, and adolescents from 13 to 18 years of age can have a normotensive blood pressure of 95-140 mm of Hg systolic and 60-90 mm of Hg diastolic. In some cases, adults can have a normotensive blood pressure of 90-119 mm of Hg systolic and 60-79 mm of Hg diastolic. In other cases, adults can be classified as having a hypotensive blood pressure with a blood pressure comprising less than 90 mm of Hg systolic pressure and less than 60 mm of Hg diastolic pressure. In yet other cases, adults can be classified as having a hypertensive blood pressure with a blood pressure comprising more than 119 mm of Hg systolic pressure and more than 79 mm of Hg diastolic pressure. In other embodiments, blood pressure varies by individual subject and classifications such as hypotensive, normotensive, and/or hypertensive can be unique to each individual subject.

In the disclosure and in the claims, the term subject (and variations thereof) may be used to refer to an individual whose blood pressure is determined by the described methods and systems. Subject can also refer to a patient and/or a person receiving treatment for a blood pressure-related condition. Subject can include an individual, a human, a mammal, or any other similar animal.

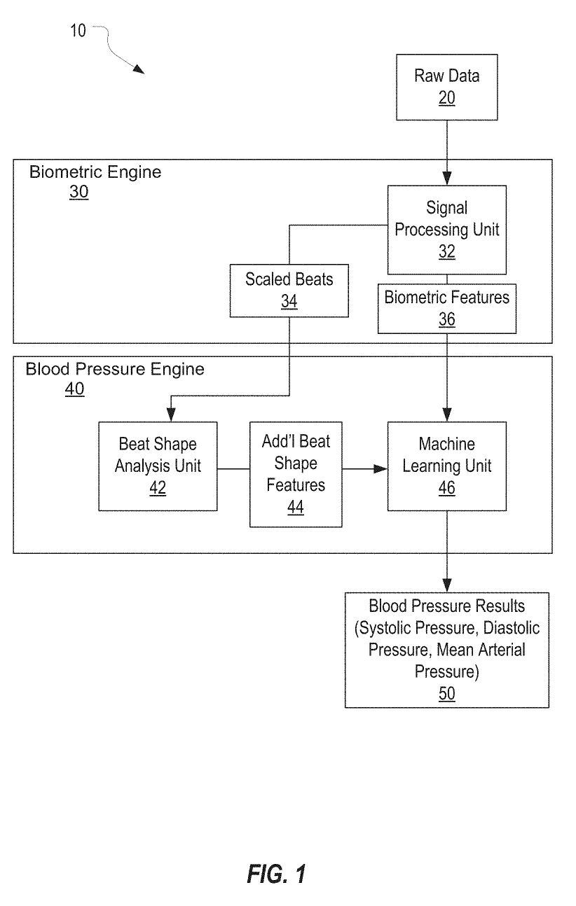

In general (and as mentioned above) some embodiments of the described methods and systems include determining a subject's blood pressure with beat shape analysis. While these methods and systems can comprise any suitable step, suitable process, suitable component or characteristic, FIG. 1 illustrates, that at least in some embodiments, they comprise a system 10 configured to process raw data to determine blood pressure. In some embodiments, system 10 comprises one or more processing modules. These processing modules can include a biometric engine 30 and a blood pressure engine 40. The biometric engine 30 can be configured to receive raw data 20. Raw data 20 can comprise raw PPG data, raw motion data captured by an accelerometer or similar device, and/or any other similar raw data related to blood pressure. While the biometric engine 30 can comprise any suitable component and/or characteristic, at least in some cases it comprises a signal processing unit 32 configured to process raw data 20. In some embodiments, the signal processing unit 32 is configured to process raw data 20 to generate scaled beats 34 and biometric features 36. Biometric features 36 can include any suitable feature, characteristic, data, or similar value related to a subject's blood pressure. In some cases, biometric features 36 can include one or more of the features listed in Table 1.

TABLE 1

| Feature Name | Feature Description |
|---|---|
| bpm | Heart Rate (beats per minute) |
| bpm.ccnt | Number of bpm clusters in associated captures, more means more shape |
| | variation |
| bpm.cnt | Number of frequency-domain lobes of bpm, 1 is normal, 0 or 2 mean |
| | multiple rates, perhaps afib |
| bpm.rng | Range of heart rates within a single capture |
| brpm | Breaths per minute (breathing rate) |
| brdb | Strength of the breadth signal (in dB) |
| stemp | Estimated skin temperature (in Celsius) |
| ir/stemp | Degree of skin temperature that correlates with ir/heat reflection |
| P | Linear correlation between RED and IR (typically more than 0.90) |
| deg.sp | Angle (in degrees) on top of systolic peak (hat angle) |
| deg.drd | Angle (in degrees) around end-diastolic ramp-down (falling angle) |
| deg.sru | Angle (in degrees) of systolic-ramp-up (rising angle) |
| delta | Frequency signature decorrelation change (how beat shapes change in |
| | frequency domain) |
| hrv | Heart Rate Variability (milliseconds) SDNN |
| mod.R | Red to IR modulation ratio |
| mod.red | Red modulation (ac/dc) |
| mod.ir | IR modulation (ac/dc) |
| spo2 | SpO₂ (peripheral capillary oxygen saturation) calculated using mod.R |
| spo2.err | Confidence interval for SpO2 estimation |
| amp.pp.pp | Pulse pressure amplitude (range of each beat in uV) |
| pp.ppv.pc | Pulse pressure variation (to adjacent beats) in percent |
| pp.vol | Normalized pulse pressure volume change from beat to beat (area under |
| | beat normalized to period) |
| pp.vol.err | Confidence interval for pp.vol estimation |
| pp.dvol | Portion of pp.vol after diastolic peak |
| pp.volv | Pulse presure volume variation |
| ppv.pc | Pulse pressure change from beat to beat (in percent) |
| ppv.pc.err | Confidence interval for ppv.pc estimation |
| amp.slope | Slope steepness in uV (microvolts) |
| amp.slope.srd | Systolic ramp-down slope in uV |
| amp.slope.red.sru | Systolic ramp-up slope in uV |
| amp.pp.red.pp | Amplitude range of red beat in uV |
| slope.sru/slope.srd | Systolic ramp-up slope device by systolic ramp-down slope |
| slope.dru/slope.drd | Systolic ramp-up slope device by diastolic ramp-down slope |
| pp.sp/dp | Pulse pressure ratio of systolic peak amplitude to diastolic peak |
| | amplitude |
| pp.sp/dn | Pulse pressure ratio of systolic peak amplitude to dicrotic notch |
| | amplitude |
| b/a | Ratio of b wave to a wave in APG/second derivation |
| d/a | Ratio of d wave to a wave in APG/second derivation (this is not as |
| | accurate as b/a) |
| i.sru | Normalized time index of systolic ramp up |
| i.sp | Normalized time index of systolic peak |
| i.srd | Normalized time index of systolic ramp down |
| i.dn | Normalized time index of dicrotic notch |
| i.dp | Normalized time index of diastolic peak |
| i.dp/i.sp | Ratio of dp to sp |
| i.dn/i.sp | Ratio of dn to sp |
| hrv.pc | Heart rate variation (in percent) |
| hrv.rms | Heart rate variation (in milliseconds) RMSSD |
| hrv.rms.err | Confidence interval (in milliseconds) of hrv.rms estimation |
| hrv.rms.low | Potentially bad heart rate variations (due to few beats or poor signal |
| | conditions) |
| hrv.rms.low.err | Confidence interval (in milliseconds) of hrv.std estimation |
| ir.offset | Amiigo wristband's IR offset value during capture |

In some embodiments, the blood pressure engine 40 is configured to receive the scaled beats 34 and the biometric features 36 from the biometric engine 30. While the blood pressure engine 40 can comprise any suitable component or characteristic, at least in some embodiments, it comprises a beat shape analysis unit 42 and a machine learning unit 46. The beat shape analysis unit 42 can be configured to analyze the scaled beats 34. The beat shape analysis unit 42 can also be configured to analyze a mean shape of the scaled beats 34. The beat shape analysis unit 42 can also be configured to generate mean beat shape data. In some cases, the beat shape analysis unit 42 can be configured to measure additional beat shape features 44 from the scaled beats 34. These additional shape features 44 can include one or more of the additional beat shape features listed in Table 2.

TABLE 2

| Name | Curves used | Description of additional beat shape feature |
|---|---|---|
| χ₁₀ | Mean shape | Scaled amplitude at the 10th landmark point |
| χ₃₅ | Mean shape | Scaled amplitude at the 35th landmark point |
| χ₁₀ + χ₃₅ | Mean shape | Sum of the scaled amplitudes |
| χ₁₀ − χ₃₅ | Mean shape | Difference of the scaled amplitudes |
| χ₁₀/χ₃₅ | Mean shape | Ratio of the scaled amplitudes |
| χ₁₀ * χ₃₅ | Mean shape | Product of the scaled amplitudes |
| αₛ | Mean shape | Scaled area under the PPG to the left of the systolic peak |
| α_{D} | Mean shape | Scaled area under the PPG to the right of the systolic peak |
| αₛ + α_{D} | Mean shape | Total scaled area under the PPG |
| αₛ − α_{D} | Mean shape | Difference of the scaled areas |
| αₛ/α_{D} | Mean shape | Ratio of the scaled areas |
| αₛ * α_{D} | Mean shape | Product of the scaled areas |
| [symbol] _{T} | Mean shape | Asymmetric score in time measured using path differences from |
| | | DTW of two sub-shapes on either side of the systolic peak |
| [symbol] _{A} | Mean shape | Asymmetric score in amplitude measured using DTW of two |
| | | sub-shapes on either side of the systolic peak |
| [symbol] _{T} + [symbol] _{A} | Mean shape | Sum of asymmetric scores |
| [symbol] _{T} − [symbol] _{A} | Mean shape | Difference of asymmetric scores |
| [symbol] _{T}/ [symbol] _{A} | Mean shape | Ratio of asymmetric scores |
| [symbol] _{T} * [symbol] _{A} | Mean shape | Product of asymmetric scores |
| [symbol] ₁ | −1 and 1 | Area between the first standard shapes in mode 1 |
| | shapes | |
| [symbol] ₂ | −1 and 1 | Area between the first standard shapes in mode 2 |
| | shapes | |
| [symbol] ₃ | −1 and 1 | Area between the first standard shapes in mode 3 |
| | shapes | |
| [symbol] ₄ | −1 and 1 | Area between the first standard shapes in mode 4 |
| | shapes | |
| [symbol] ₜₒₜₐₗ | −1 and 1 | Total area of between the first standard shapes across all modes |
| | shapes | |
| ⊥ | Mean shape | Ratio of time before systolic peak to time after systolic peak |
| Ω | Mean shape | Between the 1st standard shapes in the diastolic region versus [symbol] 1 |

In some embodiments, the machine learning unit 46 is configured to receive one or more of mean beat shape data, one or more additional shape features 44, and one or more biometric features 36. In other embodiments, the machine learning unit 46 is configured to use machine learning to develop and/or implement a predictive model for blood pressure determination. In yet other embodiments, the machine learning unit 46 is configured to use machine learning to develop and/or implement the predictive model for blood pressure determination using one or more of mean beat shape data, one or more additional shape features 44, and one or more biometric features 36. The predictive model for blood pressure determination can be implemented to determine blood pressure results 50 (e.g., systolic pressure, diastolic pressure, and/or mean arterial pressure) of the subject.

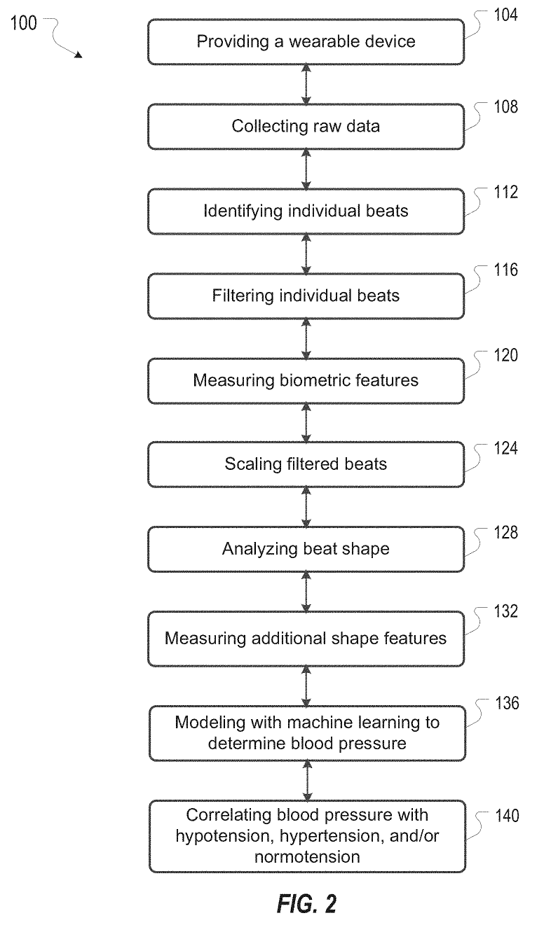

In some embodiments of the described methods and systems for determining blood pressure with beat shape analysis, the methods comprise one or more steps to acquire raw data (e.g., raw PPG data and/or raw motion data) and/or one or more steps to process the raw data to determine blood pressure. While these methods and systems can comprise any suitable step, suitable process, suitable component or characteristic, FIG. 2 illustrates, that at least in some embodiments, they can comprise a method 100 configured to acquire raw data and/or to process raw data to determine blood pressure. In some embodiments, method 100 comprises one or more of providing a wearable device 104, collecting raw data 108, identifying individual beats 112, filtering individual beats 116, measuring biometric features 120, scaling individual beats 124, analyzing beat shape 128, measuring additional shape features 132, modeling with machine learning to determine blood pressure 136, and correlating blood pressure with hypotension, hypertension, and/or normotension 140. Although a number of steps are included in method 100, any other suitable step, suitable process, suitable component or characteristic can be included in method 100. Likewise, one or more steps of method 100 can be considered as optional and method 100 can be carried out with fewer steps than described. Similarly, one or more steps of method 100 can be carried out in any order that is effective for determining blood pressure with beat shape analysis.

In some embodiments, providing a wearable device 104 comprises any suitable step or process for providing one or more wearable devices configured to be worn at least in part on a portion of a subject's body. The wearable device can be placed in any suitable portion of the subject's body effective for acquiring raw data. For example, the wearable device can be placed in any suitable portion of the subject's body effective for measuring light reflected from and/or transmitted through the portion of the subject's body. In some cases, the wearable device can be placed against a portion of the subject's skin. In other cases the wearable device can be placed on the subject's wrist, hand, finger, arm, torso, abdomen, torso, leg, foot, toe, neck, chest, back, face, ear, or any other suitable portion of the subject's body.

In some embodiments, collecting raw data 108 comprises recording raw PPG data with one or more devices configured to acquire raw PPG data and/or to process raw PPG data to determine blood pressure. In other embodiments, collecting raw data 108 comprises recording raw motion data with one or more devices configured to acquire raw motion data and/or to process raw motion data to determine blood pressure. In some instances, the devices may be configured to be wearable by the subject. In other instance, the devices may be configured to not be wearable or to only be partially wearable (e.g., a sensor portion may be wearable while the rest of the device may not be wearable).

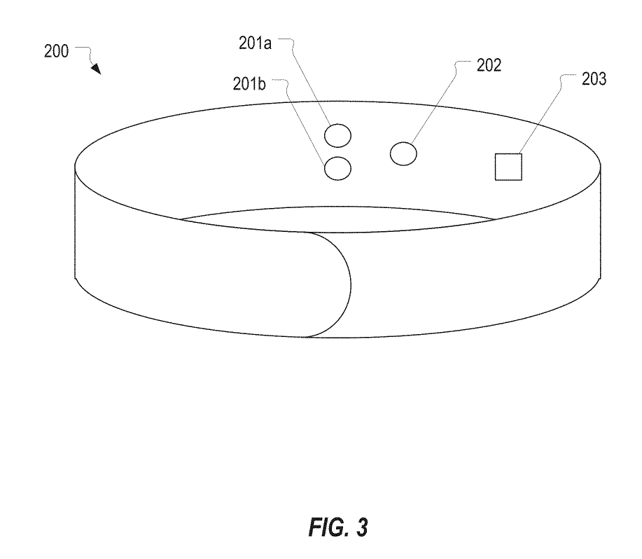

While these devices can comprise any suitable component or characteristic, FIG. 3 illustrates that, at least in some embodiments, the device comprises a bracelet 200 that is configured to acquire raw PPG data and/or to process raw PPG data to determine blood pressure. Although a bracelet configuration is described here, it is noted that other configurations of wearable devices that can be worn on the wrist or other parts of the body can also be configured to perform the described methods and systems.

Bracelet 200 can include a red light emitting diode (LED) 201a and an infrared (IR) LED 201b that are exposed on an inner surface of bracelet 200. Accordingly, when bracelet 200 is worn by a wearer, red LED 201a and IR LED 201b will emit red light and infrared waves (collectively referred to as “light”) onto the wearer's skin. The use of two separate LEDs is only an example, and a wearable sensor device may equally include only a single LED or light source or multiple LEDs or light sources. In some cases, the LEDs 201a, 201b are configured to operate at about 30 Hz.

Bracelet 200 also includes a light sensor 202 that is exposed on the inner surface of bracelet 200. Light sensor 202 is positioned adjacent LEDs 201a, 201b so as to be able to capture light (i.e., both red light and infrared waves) that is emitted by LEDs 201a, 201b and reflected from the wearer's body. Alternatively, light sensor 202 could be positioned opposite LEDs 201a, 201b so as to detect light that is transmitted through the wearer's wrist. Accordingly, the present invention extends to wearable sensor devices that include one or more LEDs and one or more light sensors for sensing light that is either transmitted through or reflected by the wearer's skin. Light sensor 202 acquires a raw PPG representing the intensity of light that it receives over time. A PPG can be generated for each of LEDs 201a, 201b. The use of a single light sensor is only an example, and a wearable sensor device may equally include multiple light sensors. The light sensor 202 can be configured to record variations in the intensity of light that it receives with respect to time. In some cases, the light sensor 202 can record variation in the intensity of light at a millisecond scale.

In some embodiments, the bracelet 200 comprises a motion sensor 203. The motion sensor 203 can be configured to acquire raw motion data of the subject. The motion sensor 203 can comprise one or more accelerometers or other suitable component to acquire raw motion data. In some cases, the motion sensor 203 comprises an accelerometer configured to collect 3-axis motion data at about 20 Hz. In other cases, the motion sensor 203 is configured to collect 3-axis motion data that is correlated in time with raw PPG data that is collected by light sensor 202.

While the raw data 20 can be collected in any suitable manner, in some embodiments, the raw data 20 is collected for a length of time at regular intervals. For example, the raw data 20 can be collected for 30 second lengths of time at intervals of 7 minutes. Raw data 20 can be collected in this manner for any suitable length of time including for minutes, for hours, for an overnight period, for days, for weeks, for months, or any other suitable length of time.

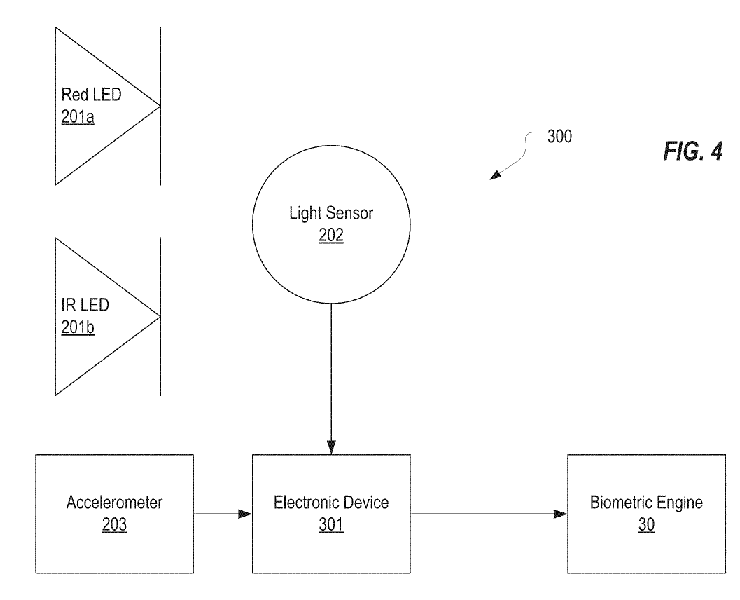

In some embodiments, identifying individual beats 112 comprises any suitable step or process for identifying individual beats within the raw PPG data. While any effective process for identifying individual beats within the raw PPG data can be used, FIG. 4 illustrates a block diagram of a circuit 300 that can be employed with bracelet 200 to identify individual beats within raw PPG data. Circuit 300 includes LEDs 201a, 201b, light sensor 202, motion sensor 203, an electronic device 301, and the biometric engine 30. The electronic device 301 can comprise any suitable electronic device such as smartphone, tablet computer, or similar device that is configured to receive raw data from the bracelet 200. The raw PPG data that is collected by the light sensor 202 can be input to the electronic device 301. The raw motion data that is collected from the motion sensor 203 can be input to the electronic device 301.

In some embodiments, the light sensor 202 and/or the motion sensor 203 are configured to wirelessly transmit raw data to the electronic device 301. In other embodiments, the electronic device 301 is configured to transmit raw data 20 to the biometric engine 30. In yet other embodiments, the electronic device 301 is configured to wirelessly transmit the raw data 20 to the biometric engine 30. In some embodiments, the electronic device 301 is configured to store the raw data 20 before transmitting to the biometric engine 30. In other embodiments the electronic device 301 is configured to process at least in part the raw data 20 before transmitting it to the biometric engine 30. In yet other embodiments, the electronic device 301 comprises the biometric engine 30.

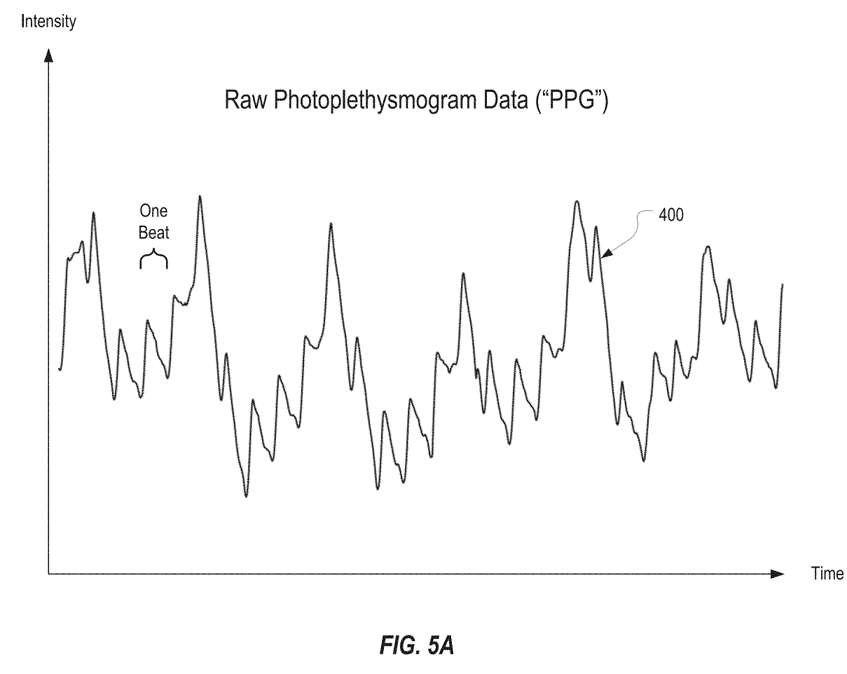

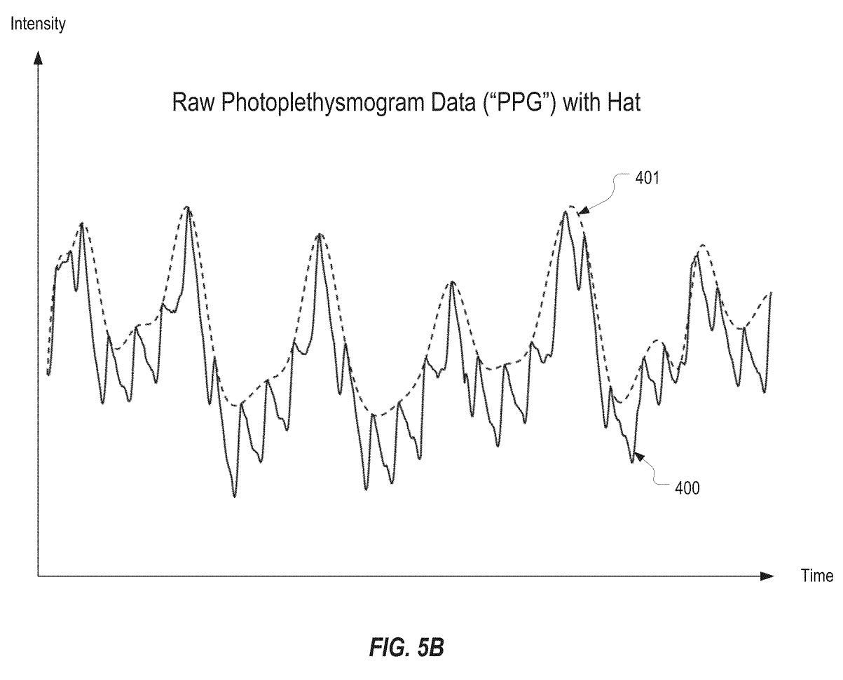

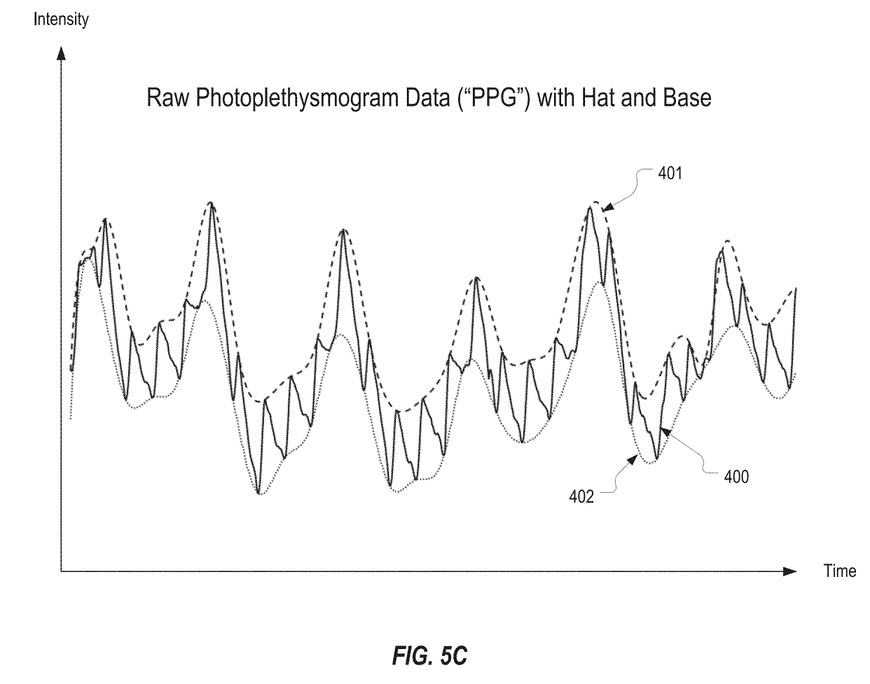

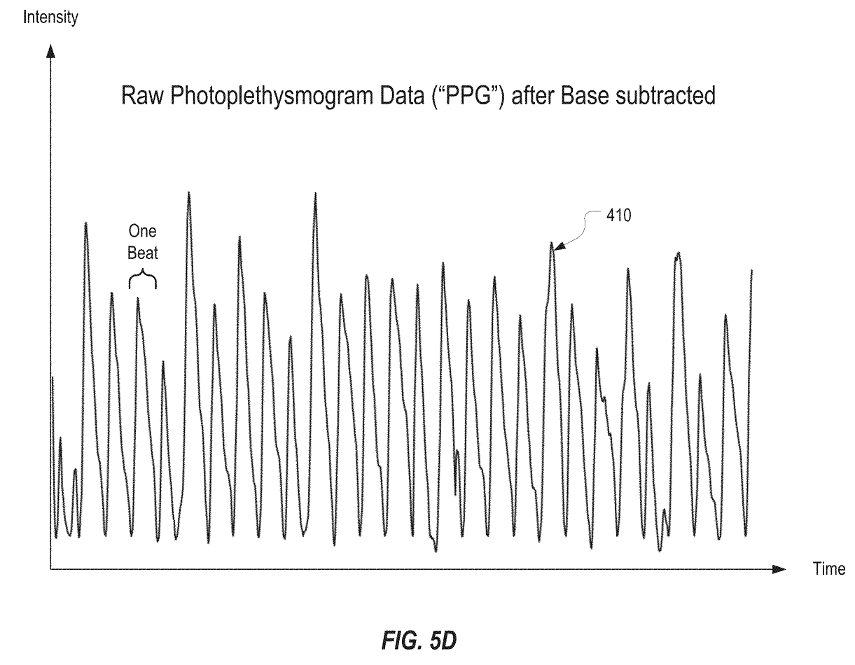

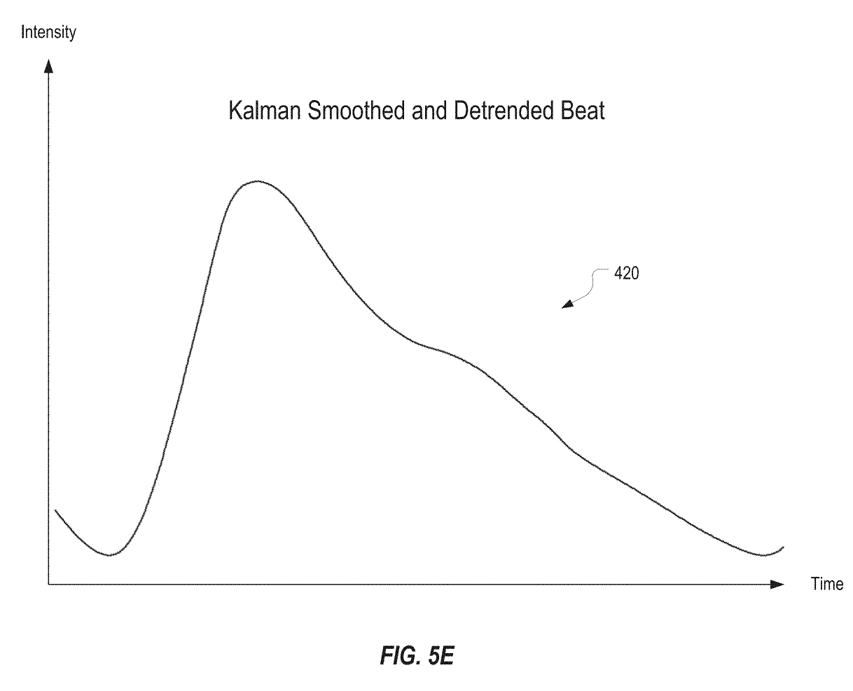

FIG. 5A illustrates a graph of an example PPG 400 that can be generated by light sensor 202 when bracelet 200 is worn. As shown, PPG 400 includes a number of individual pulse waveforms that each represents the occurrence of a heartbeat. However, these individual pulse waveforms include a significant amount of variability such as, for example, in their vertical positioning and overall shape. This variability can be caused by a number of factors including, but not limited to, for example, the breathing pattern (which primarily causes variability along the vertical axis of the PPG) or movement of the wearer. This variability in the raw PPG data can make it difficult to extract useful information from the raw PPG data.

In some embodiments, biometric engine 30 can be configured to convert the raw PPG data into beat data to facilitate the identification of individual beats. This conversion process can employ signal processing unit 32 to perform preliminary identification of peaks and valleys in the PPG. To preliminarily identify peaks in the PPG, wavelets can be employed. Wavelets are not sensitive to variations in the baseline of a signal, which variations are common in the raw PPG as shown in FIG. 5A. Wavelets are also capable of functioning on short-duration signals allowing beat data to be generated quickly when bracelet 200 is initially employed. After the peaks have been identified, the minimum value in the PPG between each peak can be identified as a valley. Accordingly, the result of this initial processing is an identification of individual beats derived from an array of peak values and an array of valley values corresponding to a particular segment of the PPG.

In some embodiments, the signal processing unit 32 is configured to perform filtering of individual beats 116 to validate peaks and valleys prior to further processing. This filtering can be performed by using a model of a human pulse waveform and/or motion data collected by the motion sensor 203. In some embodiments, filtering of individual beats can be performed using a model of a human pulse waveform that is matched to individual beats. Those individual beats that are not similar to the model can be filtered out. Any suitable statistical processes can be used to match the individual beats to the model and to determine which individual beats can be excluded. For example, if the difference between two adjacent peaks or valleys exceeds what would be reasonable in view of the model of the human pulse waveform, the corresponding portion of the PPG (or the individual beat(s)) may be excluded from further processing. Likewise, PPG data (or individual beats) that approximate the model of the human pulse waveform can be retained as valid beat data.

In some embodiments, motion data collected by the motion sensor 203 is used to filter the individual beats. In some cases, because a signal to noise ratio of the PPG deteriorates during motion of the wearer, the collected motion data can also be used to filter the individual beats that may suffer from this deteriorated signal to noise ratio. The PPG data (or individual beats) can be correlated to the motion data and PPG data (or individual beats corresponding to periods of motion can be identified. These identified peaks that correspond to periods of motion may then be excluded from further processing. In this way, PPG data (or individual beats) that are unreliable and/or imperfect are prevented from influencing later analysis of the beat data. Similarly, filtering of the individual beats within the PPG data prevents outlier data from influencing later analysis of the beat data.

In some embodiments, the step of measuring biometric features 120 comprises measuring biometric features from the filtered individual beats. For example, the filtered individual beats can be used to measure physiologic measurements such as heart rate (beats per minute (bpm)), heart rate variation, pulse volume changes, breathing rate, and SpO₂ (peripheral capillary oxygen saturation). In some cases, the filtered individual beats can be used to measure biometric features such as those listed in Table 1.

In some embodiments, the step of scaling filtered beats 124 comprises further processing of the PPG data and scaling processed individual beats to a common scale. While the further processing of the PPG data can comprise any suitable method, in some cases, the arrays of peak and valley values can be used in a three-degree polynomial to generate a hat and base respectively for the PPG. FIG. 5B illustrates an example of a hat 401 (dashed line) that was generated for PPG 400. As shown, hat 401 generally extends from peak to peak in accordance with the three-degree polynomial. FIG. 5C illustrates an example of base 402 (dotted line) that was generated for PPG 400. Similar to hat 401, base 402 extends from valley to valley in accordance with the three-degree polynomial.

As stated above, the base generally represents the effects that the subject's breathing has on the PPG. More particularly, breathing directly alters blood volume which in turn alters the amount of light that is reflected by and/or transmitted through the blood. Therefore, the effects of breathing on the PPG must be removed in order to properly extract some heartbeat characteristics from the PPG. To accomplish this, in some instances, the base can be subtracted from the PPG to yield a reliable representation of the individual pulse waveforms (or beat data 410) as shown in FIG. 5D. In some embodiments, in addition to subtracting base 402 from PPG 400 to yield beat data 410, Kalman smoothing can be performed on beat data 410 and then each beat in beat data 410 can be linearly detrended to produce a more accurate beat-shaped bellow such as is shown in FIG. 5E for one beat 420 of beat data 410.

In some embodiments, scaling individual beats to a common scale includes scaling the time component of each individual beat. For example, the time component of each individual beat (e.g., the component represented by the x-axis) can be scaled to a common scale by scaling the waveform of each individual beat to an equal number of units along the time axis. In some cases, the time component of each individual beat can be scaled to any suitable number of units along the time axis (e.g., 10 units, 100 units, 1000 units, or any other suitable value). In other cases, the time component of each individual beat can be scaled to 64 units along the time axis. In this manner, the scaled individual beats can be overlapped along the common scaled time axis.

In some embodiments, scaling individual beats to a common scale includes scaling the amplitude component of each individual beat. For example, the amplitude component of each individual beat (e.g., the component represented by the y-axis) can be scaled to a common scale by scaling the waveform of each individual beat to an equal number of units along the amplitude axis. In some cases, the amplitude component of each individual beat can be scaled to any suitable number of units along the amplitude axis (e.g., 10 units, 100 units, 1000 units, or any other suitable value). In other cases, the amplitude component of each individual beat can be scaled to a maximum of 1.0 units along the amplitude axis. In this manner, the scaled individual beats can be overlapped along the common scaled amplitude axis.

In some embodiments, analyzing beat shape 128 comprises analyzing a distinct shape of each scaled beat to determine cardiovascular values such as blood pressure. The distinct shape of each scaled beat can be defined as a quality of a configuration of data points with is invariant under a transformation such as scaling, translation, rotation, or any other similar transformation. Given that each scaled beat has a distinct shape, a variation in these distinct shapes can be modeled to determine cardiovascular values such as blood pressure.

For example, in some embodiments, beat shape analysis is performed on a collection of time-stamped scaled beat shapes (e.g., from about 2000 to about 100,000 beat shapes) captured during an interval (e.g., an overnight interval). As described above, individual beats are identified within the raw PPG data, the identified beats are filtered, and the filtered beats are scaled. The individual scaled beats can then be segregated into non-overlapping segments of a determined time length (e.g., a time length of 30 minutes) called a beat collection. In some cases, a beat collection can comprise about 50 scaled beats. In other cases, a beat collection can comprise about 30 to about 50 scaled beats. In yet other cases, a beat collection can comprise about 100 to about 200 scaled beats. In some cases, a beat collection can comprise about 30 to about 200 scaled beats.

In some embodiments, statistical methods are applied to a beat collection to learn a number of active shape models for the beat collection. These active shape models can be called beat modes. Any suitable statistical method can be applied to the beat collection to learn a beat mode including the methods described by Cootes et al. (T. F. Cootes, C. J. Taylor, D. H. Cooper, et al., “Active Shape Models—Their Training and Application,” Computer Vision and Image Understanding, 61(1): 38-59, January 1995), the disclosure of which is incorporated in its entirety. For example, Principal Component Analysis (PCA) can be used to analyze the beat collection to generate a beat mode. A first beat mode can be derived using the first eigenvalue (λi) of Principal Component Analysis (PCA), the transformation matrix T (consisting of the top P eigenvectors of PCA), and a perturbation vector b on which only the first component is allowed to vary by nλi where n={−3,−2,−1,0,1,2,3} and λi={1,2,3,4}. Likewise, additional beat modes can be derived in similar fashion (e.g., a second beat mode, a third beat mode, a fourth beat mode, and other higher beat modes).

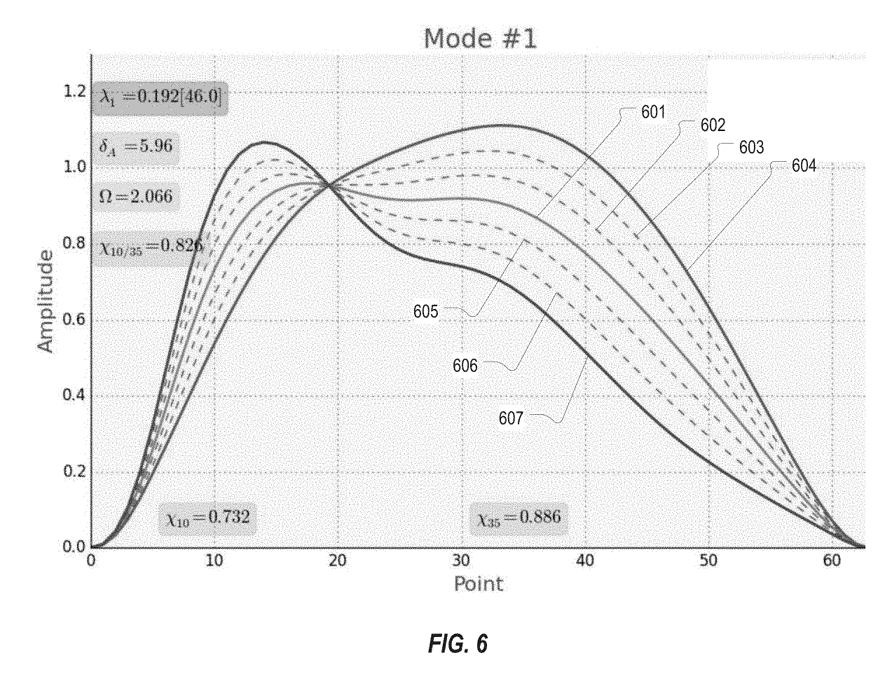

FIG. 6 illustrates some embodiments of a first beat mode. A mean beat shape 601 can be derived from the scaled beat shapes of the beat collection. In some cases, if the analysis is performed with n=0, then the mean beat shape will result. A first negative standard deviation 602 can be derived. A second negative standard deviation 603 can be derived. A third negative standard deviation 604 can be derived (e.g., −3√μ1). Similarly, one or more of a first positive standard deviation 605, a second positive standard deviation 606, and a third positive standard deviation 607 (e.g., +3√λ1) can also be derived.

In some embodiments, the first beat mode shows the variation (or the distribution) in the amplitudes of landmark points. Landmark points can include any points along the time axis that are useful for determining cardiovascular values such as blood pressure. For example, landmark points χ₁₀ and χ₃₅ can be used. In some cases, landmark points can be selected to correspond to a specific feature or portion of the beat shape (e.g., a systolic portion, a diastolic portion, or any other similar portion). In other embodiments, one or more of the mean beat shape 601, the first negative standard deviation 602, the second negative standard deviation 603, the third negative standard deviation 604, the first positive standard deviation 605, the second positive standard deviation 606, the third positive standard deviation, the landmark points, and any other suitable features are used to determine cardiovascular values such as blood pressure.

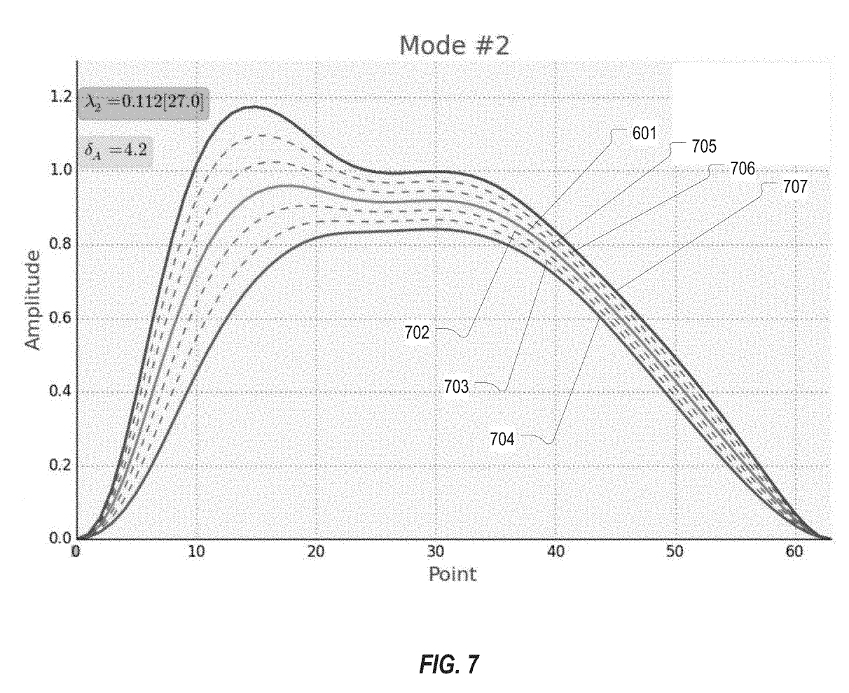

FIG. 7 illustrates some embodiments of a second beat mode. In some cases, the second beat mode can be derived from the second eigenvalue and the second eigen vector. The mean beat shape for the second beat mode is the same as the mean beat shape 601. A first negative standard deviation 702 can be derived. A second negative standard deviation 703 can be derived. A third negative standard deviation 704 can be derived (e.g., −3√λ2). Similarly, one or more of a first positive standard deviation 705, a second positive standard deviation 706, and a third positive standard deviation 707 (e.g., +3√λ2) can also be derived.

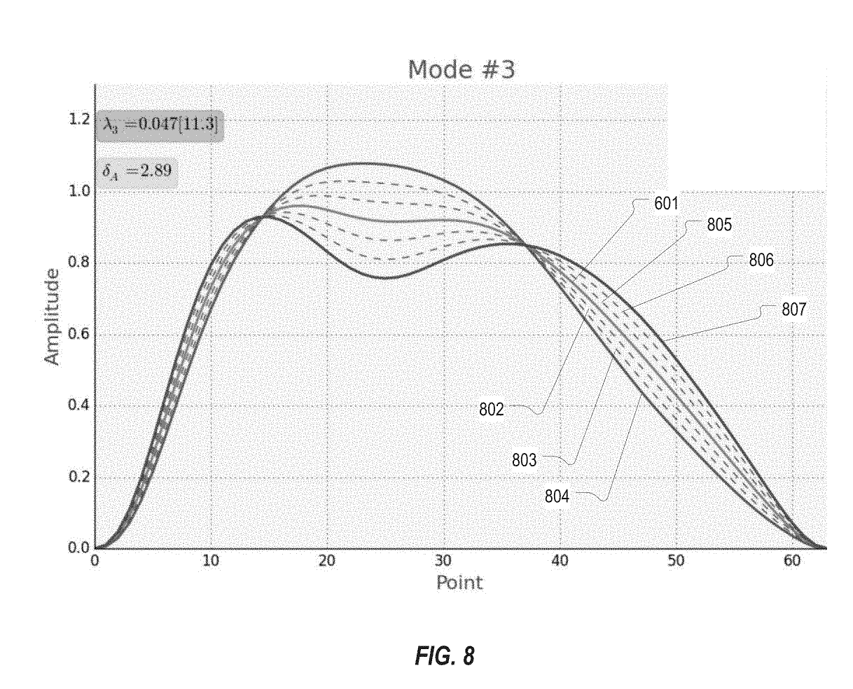

FIG. 8 illustrates some embodiments of a third beat mode. In some cases, the third beat mode can be derived from the third eigenvalue and the third eigen vector. The mean beat shape for the third beat mode is the same as the mean beat shape 601. A first negative standard deviation 802 can be derived. A second negative standard deviation 803 can be derived. A third negative standard deviation 804 can be derived (e.g., −3√λ3). Similarly, one or more of a first positive standard deviation 805, a second positive standard deviation 806, and a third positive standard deviation 807 (e.g., +3√λ3) can also be derived.

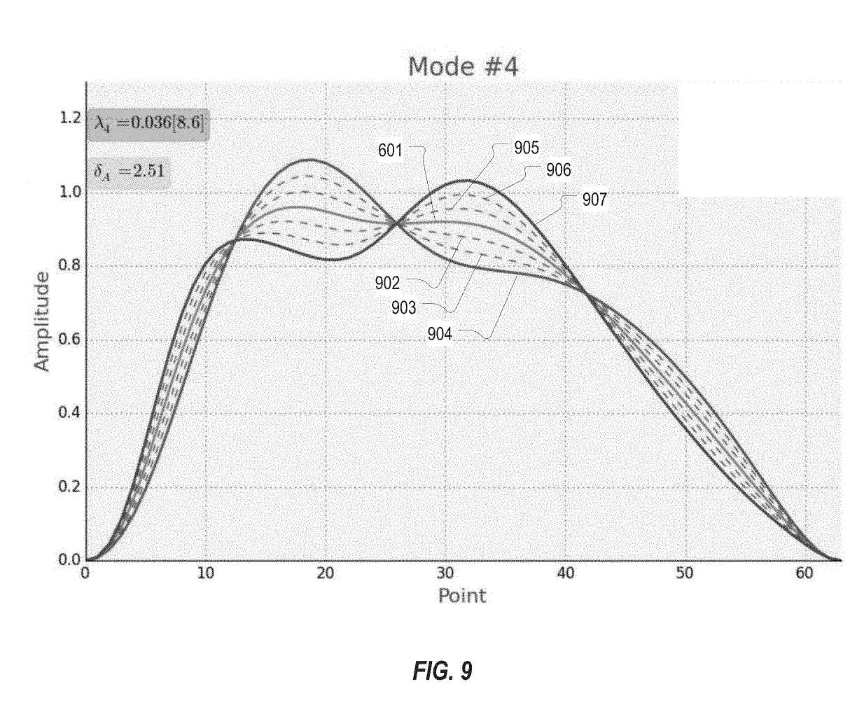

FIG. 9 illustrates some embodiments of a fourth beat mode. In some cases, the fourth beat mode can be derived from the fourth eigenvalue and the fourth eigen vector. The mean beat shape for the fourth beat mode is the same as the mean beat shape 601. A first negative standard deviation 902 can be derived. A second negative standard deviation 903 can be derived. A third negative standard deviation 904 can be derived (e.g., −3 √λ4). Similarly, one or more of a first positive standard deviation 905, a second positive standard deviation 906, and a third positive standard deviation 907 (e.g., +3√λ4) can also be derived.

In some embodiments, one or more of the features (e.g., first negative standard deviation, second negative standard deviation, third negative standard deviation, first positive standard deviation, second positive standard deviation, third positive standard deviation, landmark points, etc.) of one or more of the first beat mode, the second beat mode, the third beat mode, and the fourth beat mode are used to determine cardiovascular values such as blood pressure.

In some embodiments, measuring additional shape features 132 comprises measuring additional features from the mean beat shape and/or measuring additional features from other standard shapes. For example, measuring additional shape features 132 can comprise measuring any of the additional beat shape features listed in Table 2.

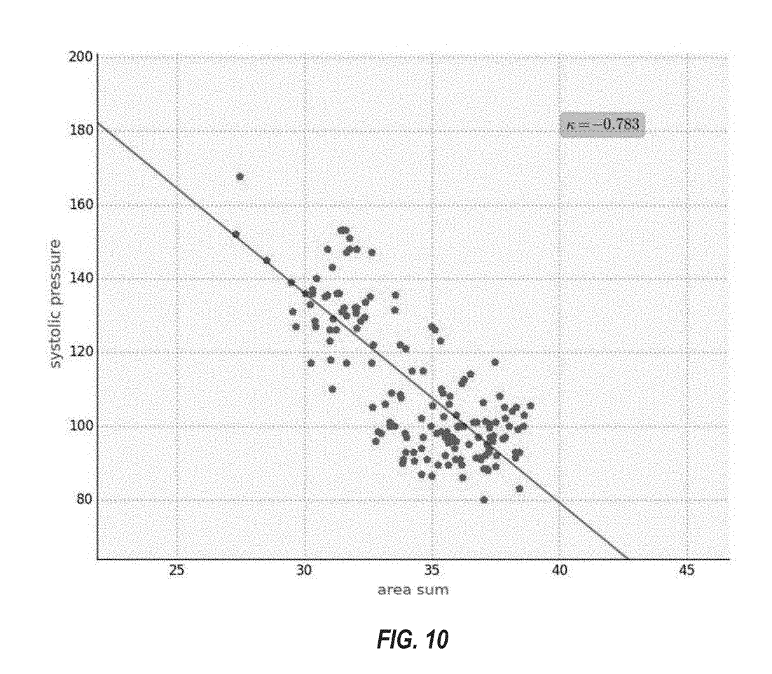

In some cases, relationships between these additional features and known blood pressures can be established. In other cases, relationship between biometric features and known blood pressures can be established. In yet other cases, these relationships can be modeled as linear relationships. The known blood pressures (e.g., ground truth blood pressure data) can be collected with conventional inflatable cuff devices at the same time as the raw data is collected. The strength of the linear relationship established between the additional features and/or biometric features and known blood pressures can be measured by a Pearson correlation coefficient (denoted by k). In other embodiments, the relationships between these additional features and/or biometric features and known blood pressures can be used to determine cardiovascular values such as blood pressure. For example, FIGS. 10-24 show relationships between some additional features and biometric features and known blood pressures.

FIGS. 10-14 shows that at least in some embodiments, linear relationships are modeled between one or more additional features and/or one or more biometric features for known systolic blood pressures. The strength of the linear relationship between the one or more additional features and/or the one or more biometric features and the known systolic blood pressure can be measured by a Pearson correlation coefficient (denoted by k). In other embodiments, the relationships between these additional features and/or biometric features and known blood pressures can be used to determine cardiovascular values such as blood pressure. Table 3 shows some of these relationships for systolic pressure that possess stronger Pearson correlation coefficients.

TABLE 3

| Feature Name | Feature Type | Correlation |
|---|---|---|
| area_sum | additional feature | −0.7825 |
| amp_sum | additional feature | −0.7204 |
| deg.sru | biometric feature | −0.7185 |
| pp.dvol | biometric feature | −0.7149 |
| i.sru | biometric feature | 0.7099 |

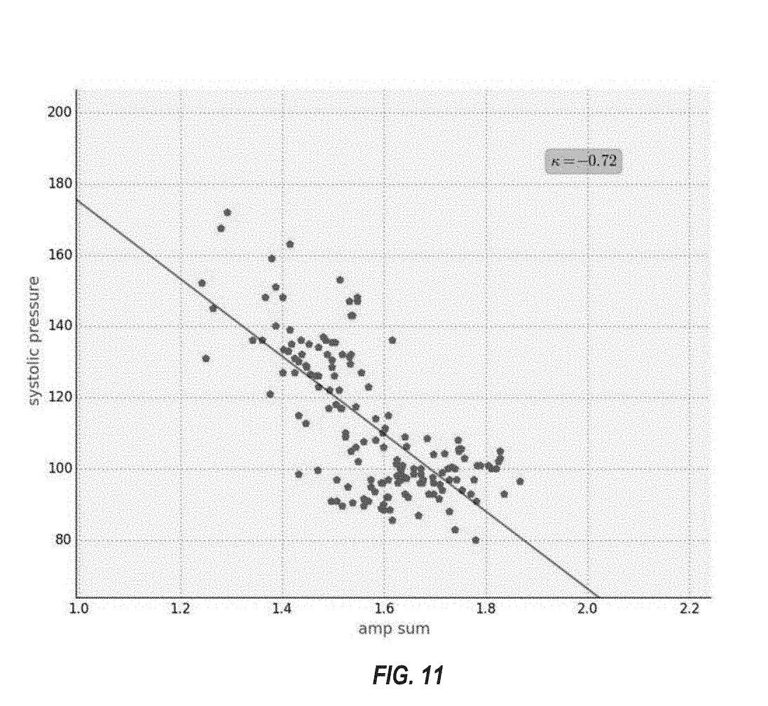

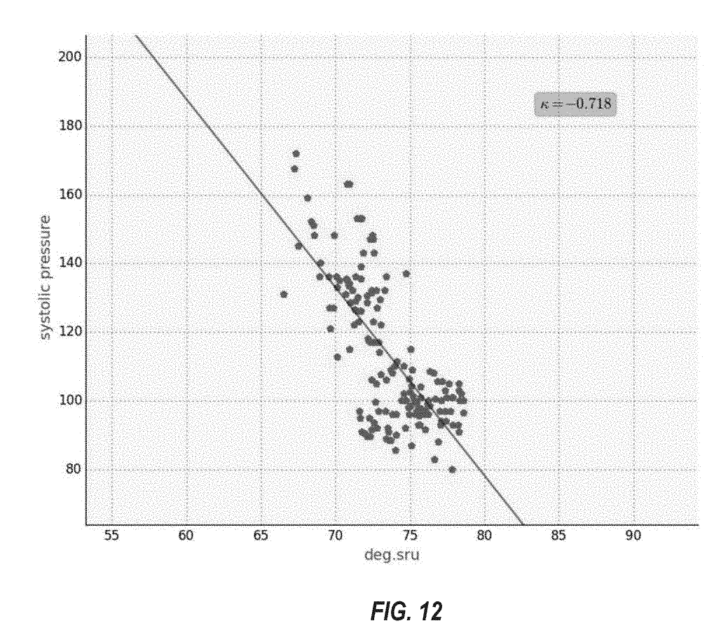

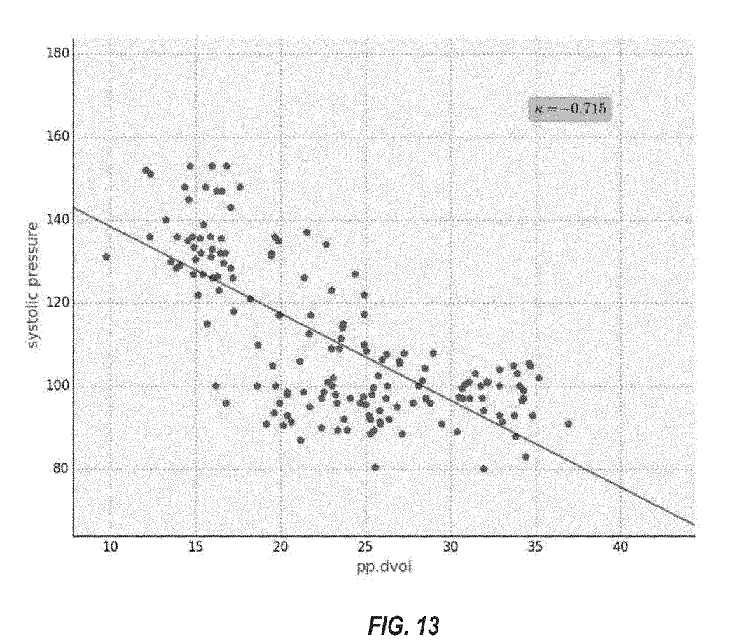

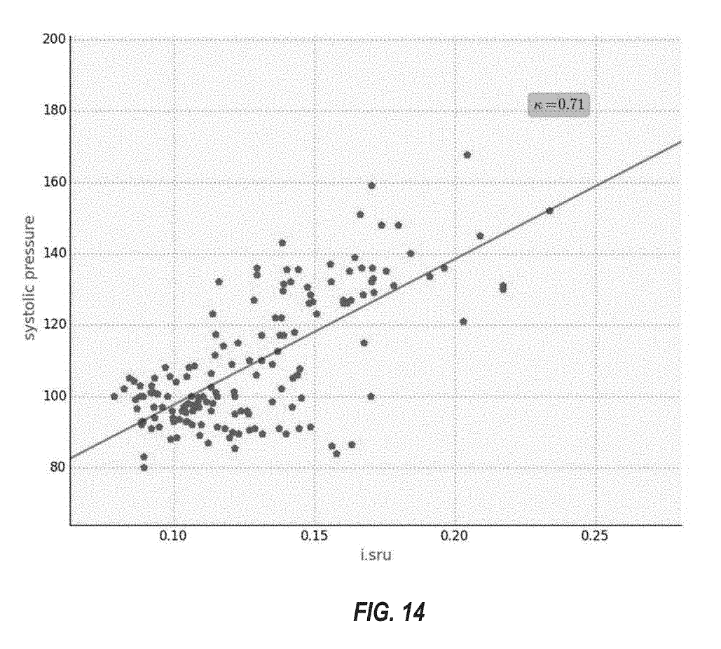

FIG. 10 illustrates a linear relationship modeled between an area_sum additional feature and a known systolic blood pressure with a Pearson correlation coefficient of −0.7825. FIG. 11 illustrates a linear relationship modeled between an amp_sum additional feature and a known systolic blood pressure with a Pearson correlation coefficient of −0.7204. FIG. 12 illustrates a linear relationship modeled between a deg.rsu biometric feature and a known systolic blood pressure with a Pearson correlation coefficient of −0.7185. FIG. 13 illustrates a linear relationship modeled between a pp.dvol biometric feature and a known systolic blood pressure with a Pearson correlation coefficient of −0.7149. FIG. 14 illustrates a linear relationship modeled between an i.sru biometric feature and a known systolic blood pressure with a Pearson correlation coefficient of 0.7099.

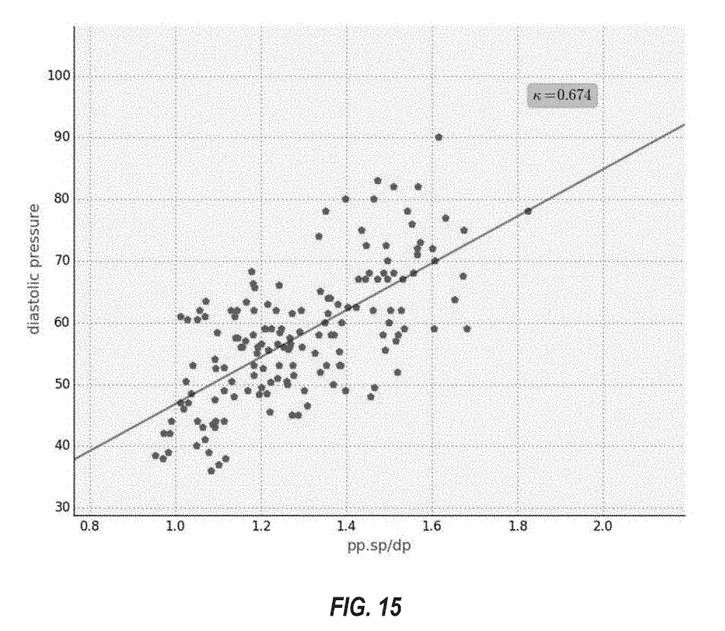

FIGS. 15-19 shows that at least in some embodiments, linear relationships are modeled between one or more additional features and/or one or more biometric features for known diastolic blood pressures. The strength of the linear relationship between the one or more additional features and/or the one or more biometric features and the known diastolic blood pressure can be measured by a Pearson correlation coefficient (denoted by k). In other embodiments, the relationships between these additional features and/or biometric features and known blood pressures can be used to determine cardiovascular values such as blood pressure. Table 4 shows some of these relationships for diastolic pressure that possess stronger Pearson correlation coefficients.

TABLE 4

| Feature Name | Feature Type | Correlation |
|---|---|---|
| pp.sp/dp | biometric feature | 0.6744 |
| area_sum | additional feature | −0.6635 |
| amp_10 | additional feature | −0.6599 |
| amp_sum | additional feature | −0.6522 |
| amp_dif | additional feature | −0.6424 |

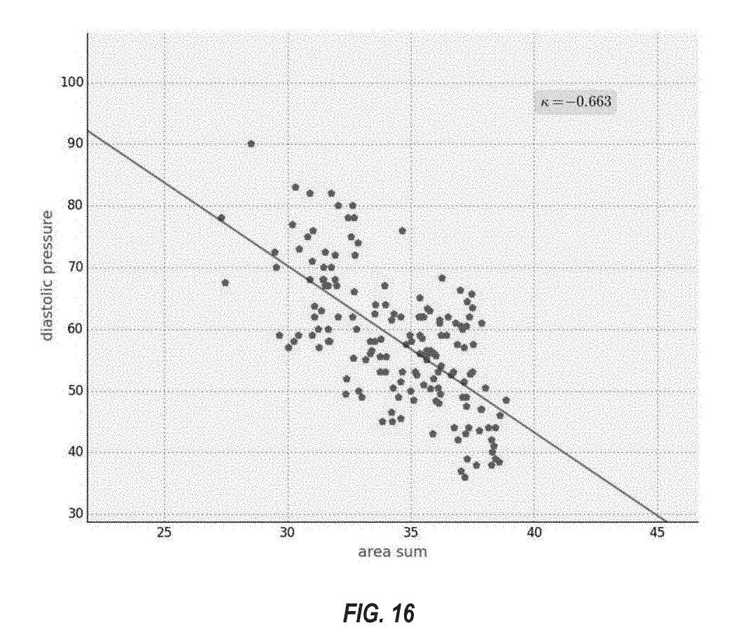

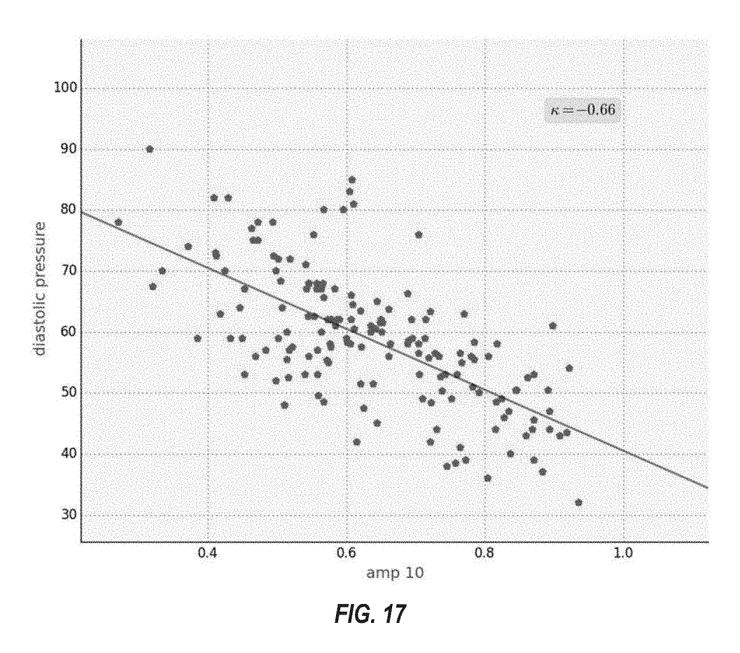

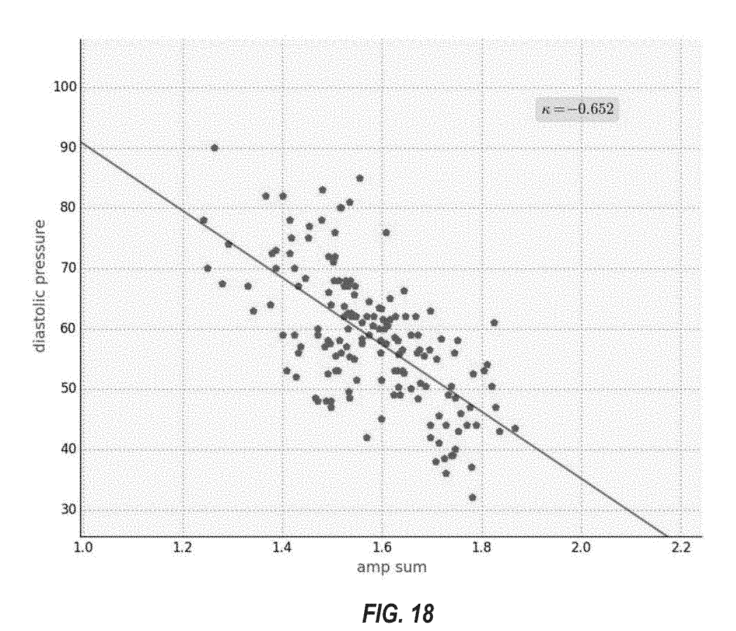

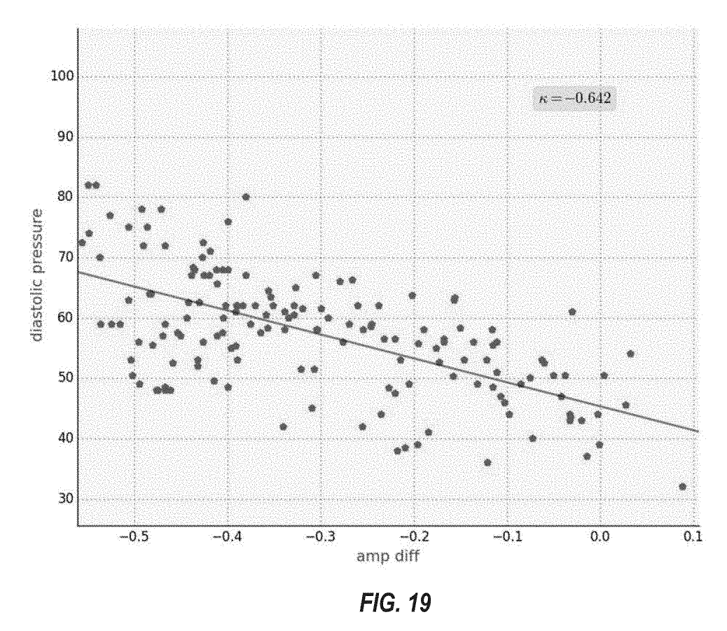

FIG. 15 illustrates a linear relationship modeled between a pp.sp/dp biometric feature and a known diastolic blood pressure with a Pearson correlation coefficient of 0.6744. FIG. 16 illustrates a linear relationship modeled between an area_sum additional feature and a known diastolic blood pressure with a Pearson correlation coefficient of −0.6635. FIG. 17 illustrates a linear relationship modeled between an amp_10 additional feature and a known diastolic blood pressure with a Pearson correlation coefficient of −0.6599. FIG. 18 illustrates a linear relationship modeled between an amp_sum additional feature and a known diastolic blood pressure with a Pearson correlation coefficient of −0.6522. FIG. 19 illustrates a linear relationship modeled between an amp_dif additional feature and a known diastolic blood pressure with a Pearson correlation coefficient of −0.6424.

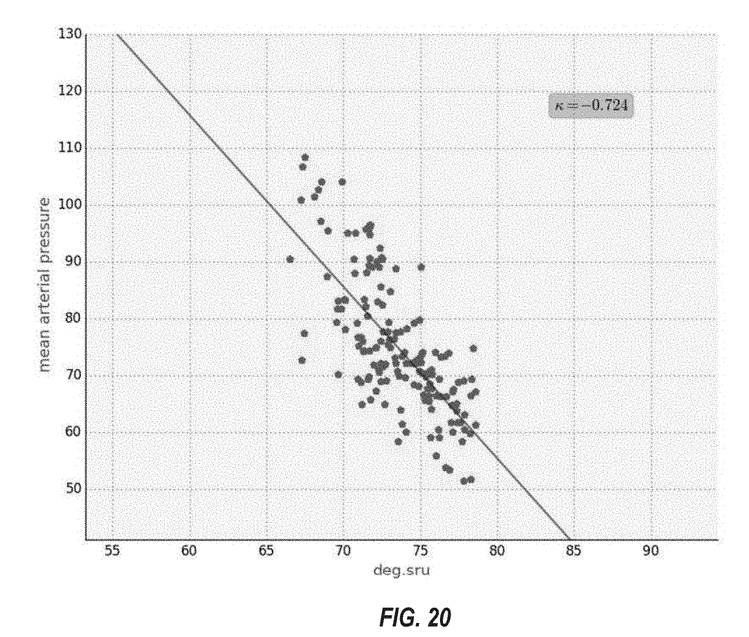

FIGS. 20-24 shows that at least in some embodiments, linear relationships are modeled between one or more additional features and/or one or more biometric features for known mean arterial pressure (MAP) blood pressures. The strength of the linear relationship between the one or more additional features and/or the one or more biometric features and the known MAP blood pressure can be measured by a Pearson correlation coefficient (denoted by k). In other embodiments, the relationships between these additional features and/or biometric features and known blood pressures can be used to determine cardiovascular values such as blood pressure. Table 5 shows some of these relationships for MAP pressure that possess stronger Pearson correlation coefficients

TABLE 5

| Feature Name | Feature Type | Correlation |
|---|---|---|
| deg.sru | biometric feature | −0.7239 |
| area_sum | additional feature | −0.7197 |
| pp.vol | biometric feature | −0.7012 |
| amp.slope.srd | biometric feature | −0.6967 |
| pp.sp/dp | biometric feature | 0.6960 |

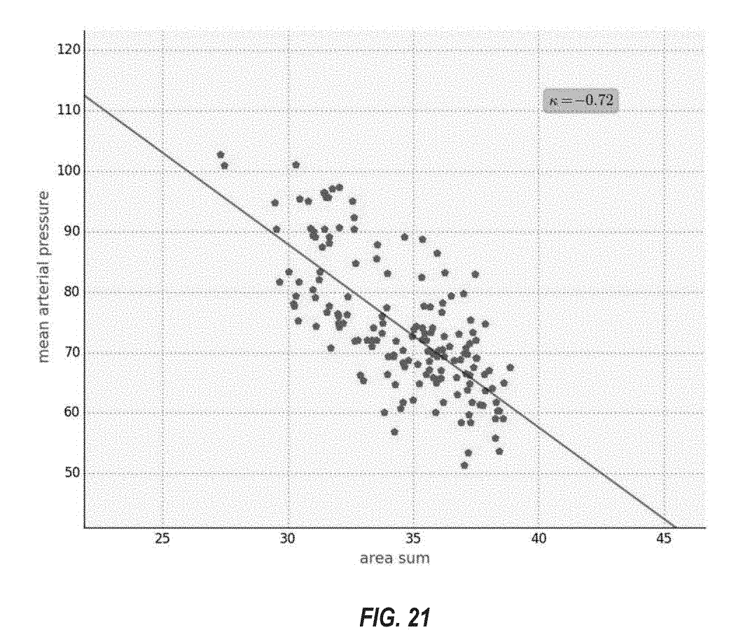

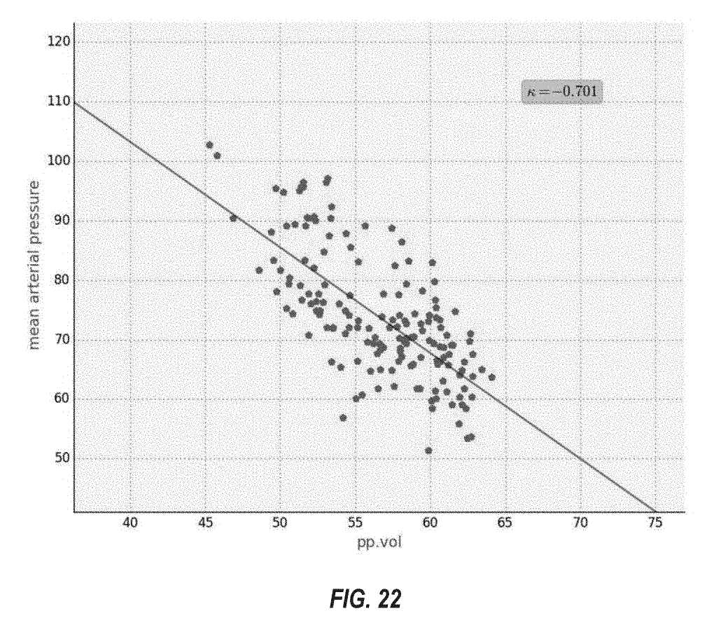

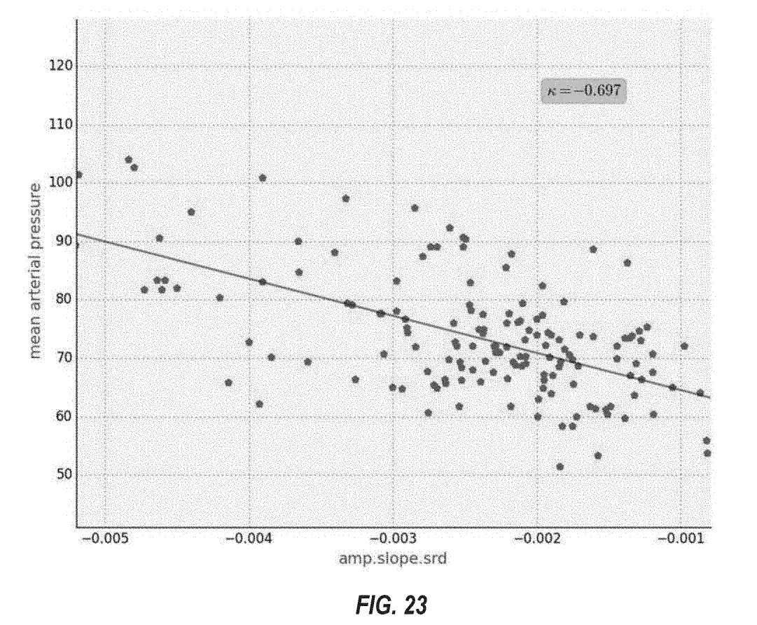

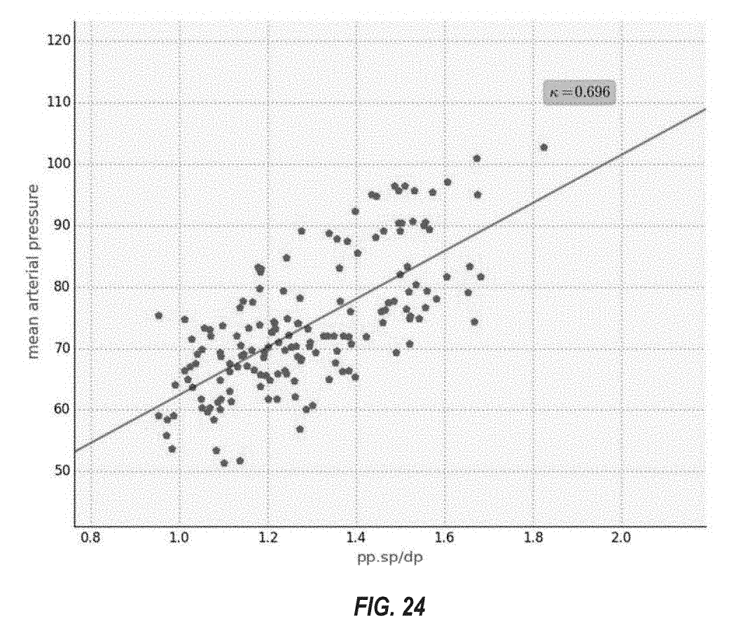

FIG. 20 illustrates a linear relationship modeled between deg.sru biometric feature and a known MAP blood pressure with a Pearson correlation coefficient of −0.7239. FIG. 21 illustrates a linear relationship modeled between an area_sum additional feature and a known MAP blood pressure with a Pearson correlation coefficient of −0.7197. FIG. 22 illustrates a linear relationship modeled between a pp.vol biometric feature and a known MAP blood pressure with a Pearson correlation coefficient of −0.7012. FIG. 23 illustrates a linear relationship modeled between an amp.slope.srd biometric feature and a known MAP blood pressure with a Pearson correlation coefficient of −0.6967. FIG. 24 illustrates a linear relationship modeled between a pp.sp/dp biometric feature and a known MAP blood pressure with a Pearson correlation coefficient of 0.6960.

In some embodiments, modeling with machine learning to determine blood pressure 136 comprises using machine learning to develop a predictive model for blood pressure. In other embodiments, modeling with machine learning to determine blood pressure 136 comprises using machine learning with one or more biometric features 36 and/or one or more additional shape features 44 to develop a predictive model for blood pressure. In yet other embodiments, modeling with machine learning to determine blood pressure 136 comprises using machine learning with one or more biometric features 36 and/or one or more additional shape features 44 and/or ground truth blood pressure data to develop a predictive model for blood pressure.

In some cases, blood pressure can be considered as a continuous variable and the predictive model for blood pressure can be modeled as a regression problem. In other cases, separate regression models for systolic pressure, diastolic pressure, and/or mean arterial pressure can be developed. In some embodiments, any suitable regression model is used to develop a predictive model for blood pressure. For example, in some instances, a linear regression model can be used to develop a predictive model for blood pressure. The linear regression model can be used to model the estimated pressure as a weighted average of selected set of biometric and additional shape features. The selected set of features includes a limited set of features to prevent zero training error (e.g., overfitting) and poor generalization performance (e.g., error rate on test datasets). Since large feature sets can lead to long computation times, a two-step feature selection approach can be used. In the first step, only features with a linear correlation (absolute value) greater than 0.5 are used. This can typically yield a pre-selected feature pool of about 20 features. In some instances, each individual blood pressure value can have a unique set of pre-selected features.

In the second step, a random subspace method can be used to select I=5 features (resulting in feature vector x) from the pre-selected feature pool and to learn a regression model p~(x) using a training dataset. The process is repeated and another set of I features are selected from the pre-selected feature pool and another model is learned with the same training set. The process is repeated to generate an ensemble of individual regression models that each predicts blood pressure. A final pressure estimate can then be estimated from a median value of the individual regression models. The 25th and 75th percentile of this ensemble of individual regression models can be used as the degree of confidence on the final pressure estimate P(x).

Individual regression models can be determined by:

$$\hat{p} ( x ) = {\sum }_{i = 0}^{I = 5} {w}_{i} {f}_{i} ( x )$$

The final pressure estimate can be determined by:

P(x)=median({\hat{p}(x)})

In some embodiments, a Multiple Adaptive Regression Splines (MARS) regression method is used to develop a predictive model for blood pressure. In some cases, the MARS regression method can be applied as described by Friedman (Friedman, Jerome H. Multivariate Adaptive Regression Splines. Ann. Statist. 19 (1991), no. 1, 1-67. doi:10.1214/aos/1176347963), the disclosure of which is incorporated in its entirety. In some instances, MARS can be used to model non-linearities using basis functions (that are piecewise linear) while considering feature interactions. In some embodiments, a pre-selection of features described above for linear regression is not performed but the random subspace method is used to pick I=5 features and learn a different MARS regression model to generate an ensemble of individual MARS regression models. A final pressure estimate can then be estimated from a median value of the individual MARS regression models.

Individual MARS regression models can be determined by:

$$\hat{p} ( x ) = {\sum }_{i = 0}^{I = 5} {w}_{i} {β}_{i} ( x )$$

In some embodiments, a Gradient Boosting Regression (GBR) regression method is used to develop a predictive model for blood pressure. In some cases, the GBR regression method can be an additive ensemble model which learns new regression trees in each step. Each learned tree can optimize the negative gradient of an arbitrary loss function. In other cases, a grid search can be used to optimize hyper-parameters of the predictive model. Similar to the MARS regression method, the pre-selection of features is not performed. In some instances, the application of random subspace method for GBR regression can also be eliminated. In other instances, the random subspace method for GBR regression can be used to improve performance. GBR regression can be used to generate a final predictive model for blood pressure that comprises an ensemble of ensembles of individual regression models.

In some embodiments, modeling with machine learning to determine blood pressure 136 comprises using machine learning to develop a predictive model for blood pressure based on raw data collected from a single wearer. For example, raw data and ground truth blood pressure data can be collected from a single individual and then used to develop a predictive model for blood pressure for the single individual. In other embodiments, modeling with machine learning to determine blood pressure 136 comprises using machine learning to develop a predictive model for blood pressure based on raw data collected from multiple wearers. In yet other embodiments, modeling with machine learning to determine blood pressure 136 comprises using machine learning to develop a predictive model for blood pressure based on raw data collected from multiple wearers based on demographic factors such as age, gender, ethnicity, vascular fitness, and other similar factors. In some embodiments, modeling with machine learning to determine blood pressure 136 comprises using machine learning to develop a single global predictive model for blood pressure that applies to any wearer.

In some embodiments, correlating blood pressure with hypotension, hypertension, and/or normotension 140 comprises using a predictive model for blood pressure to determine a wearer's blood pressure and then determining if the determined blood pressure corresponds to hypotension, hypertension, or normotension. In other embodiments, correlating blood pressure with hypotension, hypertension, and/or normotension 140 comprises using a predictive model for blood pressure derived from one or more of hypotension, hypertension, and/or normotension data and then comparing a wearer's data to the predictive model. For example, if a wearer's raw data, biometric features, and/or additional shape features are similar to a predictive model for hypertension, then the wearer's blood pressure can be correlated with hypertension. Similar comparisons can be made with predictive models for hypotension and/or normotension.

The terms “a,” “an,” “the” and similar referents used in the context of describing the disclosure (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the disclosure and does not pose a limitation on the scope of the disclosure otherwise claimed. No language in the specification should be construed as indicating any non-claimed element essential to the practice of the disclosure.

It is contemplated that numerical values, as well as other values that are recited herein are modified by the term “about”, whether expressly stated or inherently derived by the discussion of the present disclosure. As used herein, the term “about” defines the numerical boundaries of the modified values so as to include, but not be limited to, tolerances and values up to, and including the numerical value so modified. That is, numerical values can include the actual value that is expressly stated, as well as other values that are, or can be, the decimal, fractional, or other multiple of the actual value indicated, and/or described in the disclosure.

Groupings of alternative elements or embodiments of the disclosure disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this disclosure are described herein, including the best mode known to the inventors for carrying out the disclosure. Of course, variations on these described embodiments will become apparent to those of ordinary skill in the art upon reading the foregoing description. The inventor expects skilled artisans to employ such variations as appropriate, and the inventors intend for the disclosure to be practiced otherwise than specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

In closing, it is to be understood that the embodiments of the disclosure disclosed herein are illustrative of the principles of the present disclosure. Other modifications that may be employed are within the scope of the disclosure. Thus, by way of example, but not of limitation, alternative configurations of the present disclosure may be utilized in accordance with the teachings herein. Accordingly, the present disclosure is not limited to that precisely as shown and described.

We claim:

1. A method for determining a blood pressure of a subject from a photoplethysmogram, the method comprising:
acquiring raw photoplethysmogram (PPG) data from a subject by measuring light reflected from or transmitted through a portion of the subject's body;
determining a mean beat shape from the raw PPG data; and
analyzing the mean beat shape to determine a blood pressure of the subject.

2. The method of claim 1, wherein acquiring raw PPG data from a subject further comprises acquiring individual pulse wave forms on a millisecond scale.

3. The method of claim 1, wherein analyzing mean beat shape further comprises analyzing a distinct shape of the mean beat shape to determine the blood pressure.

4. The method of claim 1, further comprising identifying individual beats within the raw PPG data.

5. The method of claim 4, further comprising collecting motion data and filtering the individual beats against the motion data.

6. The method of claim 5, further comprising measuring biometric features of filtered beats.

7. The method of claim 6, further comprising scaling individual beats in time and amplitude.

8. The method of claim 7, further comprising measuring additional shape features of scaled beats.

9. A method for beat shape analysis comprising:
determining a mean beat shape from raw photoplethysmogram (PPG) data;
analyzing one or more of a biometric feature and an additional shape feature corresponding to the mean beat shape;
determining a blood pressure based at least in part on the analysis of the mean beat shape.

10. The method of claim 9, further comprising acquiring raw photoplethysmogram (PPG) data from a subject by measuring light reflected from or transmitted through a portion of the subject's body.

11. The method of claim 9, further comprising identifying individual beats within the raw PPG data by identifying peaks in the raw PPG data, identifying valleys in the raw PPG data, using the valleys to generate a base of the raw PPG data, and subtracting the base from the raw PPG data.

12. The method of claim 11, further comprising filtering of the individual beats.

13. The method of claim 11, further comprising scaling the individual beats in time and amplitude.

14. The method of claim 9, further comprising analyzing the mean beat shape by correlating biometric features corresponding to a systolic portion of the mean beat shape.

15. The method of claim 9, further comprising analyzing the mean beat shape by correlating biometric features corresponding to a diastolic portion of the mean beat shape.

16. The method of claim 9, further comprising classifying the determined blood pressure as one of hypotensive blood pressure, normotensive blood pressure, or hypertensive blood pressure.

17. A system for determining blood pressure comprising:
a wearable device configured to acquire raw photoplethysmogram (PPG) data from a subject by measuring light reflected from or transmitted through a portion of the subject's body;
a biometric engine configured to generate scaled beats and biometric features from the raw PPG data; and
a blood pressure engine configured to analyze scaled beats, biometric features, and additional beat shape features to determine the subject's blood pressure.

18. The system of claim 17, wherein the system is configured to determine the subject's blood pressure within an accuracy of 10 mm of Hg.

19. The system of claim 17, wherein the system is configured to continuously monitor the subject's blood pressure in real-time.

20. The system of claim 17, wherein the system is configured to continuously monitor increases and decreases in the subject's blood pressure.
